(19) United States
(12) Patent Application Publication
(10) Pub. No.: US 2011/0230617 A1
(43) Pub. Date: Sep. 22, 2011

(54) RADIATION-CURABLE COATING MATERIALS

(75) Inventors: Sylke Haremza, Neckargemund (DE); Michael Bueschel, Worms (DE); Tobias Johannes Korn, Frankenthal (DE); Erich Beck, Ladenburg (DE); Simon Nord, Karlsruhe (DE)

(73) Assignee: BASF SE, Ludwigshafen (DE)

(21) Appl. No.: 13/128,824

(22) PCT Filed: Nov. 11, 2009
(86) PCT No.: PCT/EP2009/064967

§ 371 Date: May 11, 2011

(30) Foreign Application Priority Data
Nov. 12, 2008 (EP) 08168882.2
Feb. 11, 2009 (EP) 09152593.1

(51) Int. Cl. C09D 4/02; C09K 3/00; C08F 2/50; C07D 209/14; B05D 7/24; B05D 3/06; B05D 3/02; B05D 3/00; B05D 5/06; B05D 7/06; B05D 7/02; B05D 7/12; B05D 7/14; B05D 7/00

(52) U.S. Cl. 524/850; 252/182.17; 522/16; 548/455; 427/521; 427/514; 427/517; 427/513

(57) ABSTRACT

The present invention relates to radiation-curable coating materials comprising new photoinitiators, and to the use thereof.

The present invention relates to radiation-curable coating materials comprising new photoinitiators, and to the use thereof.

As photoinitiators this invention uses two-component IR photoinitiator systems which comprise at least one sensitizer dye, also called sensitizer, and at least one free-radical initiator, also called coinitiator.

As a sensitizer dye, the prior art frequently uses dyes, particularly cyanine, xanthylium or thiazine dyes, and, as coinitiators, for example, boranate salts, sulfonium salts, iodonium salts, sulfones, peroxides, pyridine N-oxides or halomethyltriazines.

Cyanine dyes are composed of a cyanine cation and a corresponding anion. This can be a separate anion or else an internal anion; in other words, such that the anionic group is chemically connected to the cyanine cation. Normally they are obtained in the course of their preparation as simple salts, such as halides, tetrafluoroborates, perchlorates or tosylates, for example. Cyanine dyes with anions containing long-chain alkyl or alkyl-substituted aryl groups have not been disclosed to date. Cyanine dyes are available commercially.

Cationic cyanine dyes are frequently used in the form of their alkyl- and aryl-sulfonates, sulfates, chlorides, iodides or the like.

The sensitizers according to the present invention are selected styrylic cyanine cations featuring selected anions.

Styrylic cyanine cations are known for example from U.S. Pat. No. 6,110,987. Anions described for the styrylic cyanine cations disclosed therein, such as structures 2, 5, 10, 11, 12 and 13, for example, are halides, and, as sulfonates, only benzenesulfonate, para-toluenesulfonate and methylsulfonate are described. With the sensitizers, these have only a low solubility in coating materials.

The same applies to the styrylic cyanine cations of EP 915136 B1, of EP 1069163 A1 and of EP 1002817 B1, in each case structures 1, 3, 4 and 6 to 9 therein, which are given as salts of halides, perchlorate or tetraphenylborate. Naphthalenesulfonate as an anion is described as a counterion for other sensitizers.

The same applies to the sensitizers of EP 879829 B1 (=U.S. Pat. No. 6,165,686). EP 1170339 A2 discloses styrylic cyanine cations with organic metal complexes containing azo groups.

V. S. Jolly, P. I. Ittyerah and K. P. Sharme disclose, in Orient. J. Chem. 17(2), 275-278, 2001, styrylic cyanine cations in the form of their iodides.

Known from EP 1091247 A2, paragraphs [0109] to [0111], are long-chain aliphatic sulfonates as counterions for cyanine cations.

Long-chain sulfonates as anions are known for example from WO 2006/058731. Following exposure, however, the sensitizers disclosed as cyanine cations therein have a coloredness which is disruptive especially in clearcoat materials.

It was an object of the present invention to provide sensitizers for radiation-curable coating materials that exhibit effective through-curing on exposure, have a low level of coloredness after curing, and exhibit good solubility in radiation-curable coating materials.

This object has been achieved by means of sensitizer systems (A) for radiation-curable coating materials, comprising a styrylic cation D⁺ of the formula (I)

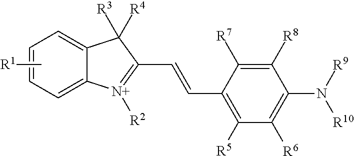

and as counterion an anion An⁻selected from the group consisting of anions of the formula (II)

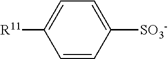

and alkyl sulfates of the formula (III)

R¹²—O—SO₂—O^{e}

in which

R¹, R⁵, R⁶, R⁷ and R⁸ each independently can be hydrogen, C₁-C₁₈ alkyl or C₁-C₁₈ alkyloxy,

R¹ can additionally be halogen, preferably bromine

R², R³ and R⁴ each independently can be C₁-C₁₈ alkyl,

R⁹ and R¹⁰ each independently can be unsubstituted or aryl-, alkyl-, aryloxy-, alkyloxy-, heteroatom- and/or heterocycle-substituted C₁-C₁₈ alkyl, C₆-C₁₂ aryl or C₅-C₁₂ cycloalkyl,

R¹¹ can be C₅-C₁₈ alkyl and

R¹² can be C₁-C₁₈ alkyl.

Definitions therein are as follows:

C₁-C₁₈ alkyl optionally substituted by aryl, alkyl, aryloxy, alkyloxy, heteroatoms and/or heterocycles is for example methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, heptadecyl, octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl,1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl,

unsubstituted or aryl-, alkyl-, aryloxy-, alkyloxy-, heteroatom- and/or heterocycle-substituted C₅-C₁₈ alkyl is for example pentyl, hexyl, heptyl, octyl, 2-ethylhexyl, 2,4,4-trimethylpentyl, decyl, dodecyl, tetradecyl, heptadecyl, octadecyl, 1,1-dimethylpropyl, 1,1-dimethylbutyl, 1,1,3,3-tetramethylbutyl,

C₆-C₁₂ aryl optionally substituted by aryl, alkyl, aryloxy, alkyloxy, heteroatoms and/or heterocycles is for example phenyl, tolyl, xylyl, α-naphthyl, β-naphthyl, 4-biphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl,

C₅-C₁₂ cycloalkyl optionally substituted by aryl, alkyl, aryloxy, alkyloxy, heteroatoms and/or heterocycles is for example cyclopentyl, cyclohexyl, cyclooctyl, cyclododecyl, methylcyclopentyl, dimethylcyclopentyl, methylcyclohexyl, dimethylcyclohexyl, diethylcyciohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl, for example.

The radicals R¹, R⁵, R⁶, R⁷ and R⁸ are each independently preferably hydrogen or C₁ to C₄ alkyl, more preferably hydrogen, methyl or ethyl, very preferably hydrogen or methyl, and especially hydrogen.

C₁ to C₄ alkyl in this specification is methyl, ethyl, propyl, isopropyl, n-butyl, sec-butyl or tert-butyl, preferably methyl, ethyl or n-butyl, more preferably methyl or ethyl and very preferably methyl.

The radical R¹ is located on the indole ring preferably in position 5 or 6.

Of the radicals R⁵, R⁶, R⁷ and R⁸, preferably at least two are hydrogen, more preferably at least 3, and very preferably all four are hydrogen.

The radical R² may preferably be C₁ to C₄ alkyl, more preferably methyl or ethyl, and very preferably ethyl.

The radicals R³ and R⁴ may each independently be preferably methyl or ethyl, more preferably ethyl. In one preferred embodiment the two radicals R³ and R⁴ are alike.

The radicals R⁹ and R¹⁰ are each independently preferably unsubstituted or aryl-, alkyl-, aryloxy-, alkyloxy-, heteroatom- and/or heterocycle-substituted C₁-C₁₈ alkyl and more preferably are methyl, ethyl, n-propyl, 2-hydroxyethyl, 2-hydroxypropyl, 2-chloroethyl, 2-cyanoethyl, 2-acetoxyethyl, cyclohexyl or cyclopentyl, very preferably methyl, ethyl, 2-hydroxyethyl, 2-chloroethyl and 2-cyanoethyl, especially methyl, ethyl and 2-cyanoethyl.

In one possible embodiment the radicals R⁹ and R¹⁰ may form a joint chain, as for example 1,5-pentylene, 1,4-butylene or 3-oxa-1,5-pentylene.

In a further possible embodiment the radicals R⁹ and R⁸ and/or R⁶ and R¹⁰ may form a joint chain, as for example 1,2-ethylene or 1,3-propylene. In this case preferably both the radicals R⁹ and R⁸ and the radicals R⁶ and R¹⁸ form a chain, more preferably in each case of the same chain length.

The radical R¹¹ is for example a linear or branched alkyl group, preferably a linear alkyl group. Preferably it is 1-pentyl, 1-hexyl, 2-ethyl-1-hexyl, 1-octyl, 1-nonyl, 1-decyl, 1-undecyl, 1-dodecyl or 1-tetradecyl. Particular preference is given to 1-hexyl, 1-octyl, 1-decyl, 1-dodecyl or 1-tetradecyl.

The radical R¹² may for example be methyl, ethyl, n-butyl, n-hexyl, n-octyl, n-decyl or n-dodecyl, preferably methyl, ethyl, n-butyl, or n-dodecyl and more preferably methyl, ethyl or n-dodecyl.

The styrylic cyanine cation D⁺ is preferably selected from the group consisting of the following individuals:

| | | | | | | | | | | |
|---|---|---|---|---|---|---|---|---|---|---|
| No. | R¹ | R² | R³ | R⁴ | R⁵ | R⁶ | R⁷ | R⁸ | R⁹ | R¹⁰ |

| 1 | H | Ethyl | Methyl | Methyl | H | H | H | H | R⁹ + R¹⁰: 1,4-Butylene | |
|---|---|---|---|---|---|---|---|---|---|---|
| 2 | H | Ethyl | Methyl | Methyl | H | * | H | * | * | * |
| 3 | H | Ethyl | Methyl | Methyl | H | H | H | H | 2-Chloroethyl | 2-Chloroethyl |
| 4 | H | Ethyl | Methyl | Methyl | H | H | H | H | 2-Hydroxy- | Methyl |
| | | | | | | | | | ethyl | |
| 5 | H | Ethyl | Methyl | Methyl | H | H | H | H | 2-Hydroxy- | 2-Hydroxy- |
| | | | | | | | | | ethyl | ethyl |
| 6 | H | Ethyl | Methyl | Methyl | H | H | H | H | 2-Hydroxy- | Cyclohexyl |
| | | | | | | | | | ethyl | |
| 7 | H | Ethyl | Methyl | Methyl | H | H | H | H | 2-Hydroxy- | 2-Cyanoethyl |
| | | | | | | | | | ethyl | |
| 8 | H | Ethyl | Methyl | Methyl | H | H | H | H | 2-Acetoxy- | 2-Acetoxy- |
| | | | | | | | | | ethyl | ethyl |
| 9 | H | Ethyl | Methyl | Methyl | H | H | H | H | 2-Cyanoethyl | Methyl |
| 10 | H | Ethyl | Methyl | Methyl | Methyl | H | H | H | 2-Hydroxy- | 2-Cyanoethyl |
| | | | | | | | | | ethyl | |
| 11 | H | Ethyl | Methyl | Methyl | Methyl | H | H | H | Ethyl | Ethyl |
| 12 | H | Ethyl | Methyl | Methyl | Methyl | H | H | H | Methyl | Methyl |
| 13 | H | Ethyl | Methyl | Methyl | Methyl | H | H | H | R⁹ + R¹⁰: 1,5-Pentylene | |
| 14 | H | Ethyl | Methyl | Methyl | Methyl | H | H | H | R⁹ + R¹⁰: 3-Oxa-1,5- | |
| | | | | | | | | | pentylene | |
| 15 | H | Ethyl | Methyl | Methyl | H | H | H | H | R⁹ + R¹⁰: 3-Oxa-1,5- | |
| | | | | | | | | | pentylene | |
| 16 | H | Ethyl | Methyl | Methyl | H | H | H | H | Methyl | Methyl |
| 17 | H | Methyl | Methyl | Methyl | H | H | H | H | Methyl | 2-Chloroethyl |
| 18 | H | Methyl | Methyl | Methyl | Methyl | H | H | H | Ethyl | 2-Chloroethyl |
| 19 | H | Ethyl | Methyl | Methyl | H | H | H | H | 2-Cyanoethyl | 2-Cyanoethyl |
| 20 | H | Methyl | Methyl | Methyl | H | H | H | H | 2-Cyanoethyl | 2-Cyanoethyl |

* No. 2; R⁹ + R⁸: 1,3-propylene, R¹⁰ + R⁶: 1,3-propylene

Preferably the anion An⁻ is of the formula (II), more preferably n-octylsulfonate, n-decylsulfonate or n-dodecylsulfonate, and also 4-alkylbenzenesulfonates with alkyl radicals composed of 6 to 12 carbon atoms, such as, for example, 4-hexylbenzenesulfonate, 4-octylbenzenesulfonate, 4-decylbenzenesulfonate or 4-dodecylbenzenesulfonate. In this context the products in question may, in a way which is known in principle, also be technical products which exhibit a distribution of different alkyl radicals with different lengths. Particular preference as An⁻is given to 4-n-dodecylbenzenesulfonate.

The sensitizer is used preferably in a mixture with a coinitiator (B) of the formula (IV)

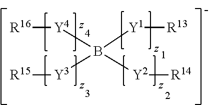

having an associated counterion 1/x cat^{x+},

in which

x is 1 or 2,

cat is a cation,

z¹, z², z³ and z⁴ independently are each 0 or 1,

the sum z¹+z²+z³+z⁴being 0, 1, 2 or 3, preferably 0, 1 or 2, more preferably 0 or 1, and very preferably 0,

Y¹, Y², Y³ and Y⁴ independently are each O, S or NR¹⁷,

R¹³, R¹⁴, R¹⁵ and R¹⁶ each independently are C₁-C₁₈ alkyl, C₂-C₁₈ alkyl, which is uninterrupted or interrupted by one or more oxygen and/or sulfur atoms and/or by one or more substituted or unsubstituted imino groups, or are C₆-C₁₂ aryl, C₅-C₁₂ cycloalkyl or a five- to seven-membered heterocycle containing oxygen, nitrogen and/or sulfur atoms, it being possible for the stated radicals each to be substituted by aryl, alkyl, aryloxy, alkyloxy, heteroatoms and/or heterocycles, and

R¹⁷ is hydrogen, C₁-C₁₈ alkyl or C₆-C₁₂ aryl

with the proviso that at least one of the radicals R¹³ to R¹⁶ is a C₁-C₁₈ alkyl radical, and at least one of the radicals R¹³ to R¹⁶ is a C₆-C₁₂ aryl radical, it being possible for the stated radicals to be substituted in each case by aryl, alkyl, aryloxy, alkyloxy, heteroatoms and/or heterocycles.

Y¹, Y², Y³ and Y⁴ independently of one another are each preferably oxygen or NR¹⁷ and more preferably oxygen.

R¹⁷ is preferably hydrogen or C₁-C₄ alkyl.

R¹³, R¹⁴, R¹⁵ and R¹⁶ are preferably independently of one another in each case C₁-C₁₈ alkyl, C6-C₁₂ aryl or C₅-C₁₂ cycloalkyl, more preferably C₁-C₁₈ alkyl and C₆-C₁₂ aryl, very preferably selected from the group consisting of methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, heptadecyl, octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl,1-(p-butylphenyl)ethyl, phenyl, tolyl, xylyl, α-naphthyl, β-naphthyl, 4-biphenylyl, methylphenyl, dimethylphenyl, trimethylphenyl, ethylphenyl, diethylphenyl, isopropylphenyl, tert-butylphenyl and dodecyl phenyl, selected in particular from the group consisting of methyl, ethyl, propyl, n-butyl, hexyl, octyl, 2-ethylhexyl, dodecyl, benzyl, 2-phenylethyl, phenyl, tolyl, α-naphthyl and β-naphthyl, and especially selected from the group consisting of methyl, ethyl, n-propyl, n-butyl, benzyl, phenyl and tolyl.

In accordance with the invention, at least one of the radicals R¹³ to R¹⁶ is C₁-C₁₈ alkyl and at least one is C₆-C₁₂ aryl; preferably at least one is C₁-C₁₈ alkyl and at least one is C₆-C₁₂ aryl and the other two are likewise selected from the group comprising C₁-C₁₈ alkyl and C₆-C₁₂ aryl; more preferably at least one is C₁-C₁₈ alkyl and at least two are C₆-C₁₂ aryl; and very preferably one is C₁-C₁₈ alkyl and three are C₆-C₁₂ aryl.

The amount of sensitizer dye (A) in the radiation-curable coating material is chosen by the skilled worker so as to obtain sufficient photocuring of the coating material. As a general rule, an amount of less than 5% by weight is sufficient. An amount which has proven particularly appropriate is from 0.05% to 4% by weight, relative to the sum of all of the components of the coating material, preferably 0.1% to 3%, more preferably 0.2% to 2.5%, and very preferably 0.3% to 2.0% by weight. In accordance with the invention it must be ensured that added sensitizer dye is fully dissolved in the coating material.

The solubility of the sensitizer dye in the coating material is preferably at least 0.2%, more preferably at least 0.5%, very preferably at least 1.0% and, for example, at least 2% by weight.

In accordance with the invention the mixtures of the invention likewise comprise a component (B) comprising an anionic boron compound of the formula (IV).

These anionic boron compounds possess as their counterion an x-tuply positively charged cation catx+. Such cations may be, for example, alkali metal, alkaline earth metal or ammonium ions, e.g., Mg²⁺, Li⁺, Na⁺ or K⁺, but are preferably ammonium ions.

Ammonium ions for the purposes of the present invention are ionic compounds which comprise at least one tetrasubstituted nitrogen atom, the substituents being selected from C₁-C₁₈ alkyl and C₆-C₁₂ aryl and being preferably alkyl radicals. It is of course also possible for two or more substituents to be linked to form a ring, so that the quaternary nitrogen atom is part of a five- to seven-membered ring.

Examples of ammonium cations are tetra-n-octylammonium, tetramethylammonium, tetraethylammonium, tetra-n-butylammonium, trimethylbenzylammonium, trimethylcetylammonium, triethylbenzylammonium, tri-n-butylbenzylammonium, trimethylethylammonium, tri-n-butylethylammonium, triethylmethylammonium, tri-n-butylmethylammonium, diisopropyldiethylammonium, diisopropylethylmethylammonium, diisopropylethylbenzylammonium, N,N-dimethylpiperidinium, N,N-dimethylmorpholinium, N,N-dimethylpiperazinium or N-methyldiazabicyclo[2.2.2]octane. Preferred alkylammonium ions are tetraoctylammonium, tetramethylammonium, tetraethylammonium and tetra-n-butylammonium, particular preference is given to tetraoctylammonium and tetra-n-butylammonium, and tetra-n-butylammonium is especially preferred.

Examples of ammonium ions comprising ring systems are methylated, ethylated, n-butylated, cetylated or benzylated piperazines, piperidines, imidazoles, morpholines, quinuclidines, quinolines, pyridines or triethylenediamines.

In one preferred embodiment the cations cat^{x+} of the anionic boron compound are cations of the kind described in WO 2008/058885 A2, page 16, line 30 to page 25, line 29. These passages are hereby made part of the present disclosure content.

Particular preference is given to those cations cat^{x+} selected from the group consisting of 1-methylimidazolium, 1-butylimidazolium, 1,3-dimethylimidazolium, 1,2,3-trimethyl-imidazolium, 1-n-butyl-3-methylimidazolium, 1-ethyl-3-methylimidazolium, 1,3,4,5-tetra-methylimidazolium, 1,3,4-trimethylimidazolium, 2,3-dimethylimidazolium, 1-butyl-2,3-dimethylimidazolium, 3,4-dimethylimidazolium, 2-ethyl-3,4-dimethylimidazolium, 3-methyl-2-ethylimidazolium, 3-butyl-1-methylimidazolium, 3-ethyl-1-methylimidazolium, 3-butyl-1-ethylimidazolium, 3-butyl-1,2-dimethylimidazolium, 1,3-di-n-butylimidazolium, 3-butyl-1,4,5-trimethylimidazolium, 3-butyl-1,4-dimethylimidazolium, 3-butyl-2-methylimidazolium, 1,3-dibutyl-2-methylimidazolium, 3-butyl-4-methylimidazolium, 3-butyl-2-ethyl-4-methylimidazolium, 3-butyl-2-ethylimidazolium, 1-methyl-3-octylimidazolium and 1-decyl-3-methylimidazolium.

Particular preference as cat^{x+} is given to 1-methylimidazolium, 1-butylimidazolium, 1-butyl-4-methylpyridinium, 1-n-butyl-3-methylimidazolium, 1-ethyl-3-methylimidazolium and 1-n-butyl-3-ethylimidazolium.

The present invention further provides mixtures of components (A) and (B) that can be used as photoinitiators:

The mixtures of the invention comprise
at least one component (A) of the formula An⁻ Cya⁺, as indicated above, and
at least one component (B), preferably of the formula (IV), having a counterion 1/x cat^{x+}.

The mixtures of the invention may also comprise, additionally, sulfonium salts, iodonium salts, sulfones, peroxides, pyridine N-oxides or halomethyltriazines as their sensitizer dye.

Suitable sulfonium salts are described for example in DE-A1 197 30 498, particularly on page 3 therein, in lines 28-39, that passage being hereby expressly incorporated into the present disclosure content by reference.

These salts are preferably salts of the formula

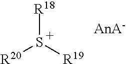

in which

R¹⁸ and R¹⁹ are each an optionally substituted aryl group and

R²⁰ is an optionally substituted alkyl group, an optionally substituted alkenyl group, an optionally substituted alicyclic group, an optionally substituted aryl group or an optionally substituted aralkyl group, and AnA⁻ is an anion.

Particular preference is given to triphenylsulfonium, diphenylanisylsulfonium, diphenyl(4-tolyl)sulfonium, diphenyl(4-fluorophenyl)sulfonium, diphenyl[4-(phenyl-thio)phenyl)sulfonium, diphenylbenzylsulfonium, diphenyl(4-chlorobenzyl)sulfonium, diphenyl(4-bromobenzyl)sulfonium, diphenyl(4-cyanobenzyl)sulfonium, di(4-tert-butylphenyl)benzylsulfonium, dianisyl(4-bromophenyl)sulfonium, diphenylphenacylsulfonium, diphenyl(4-chlorophenacyl)sulfonium, diphenyl(4-cyanophenacyl)sulfonium, diphenylallylsulfonium, diphenylmesylsulfonium, diphenyl-p-toluenesulfonylmethylsulfonium, diphenyl(dimethylsulfoniumylmethyl)sulfonium and diphenyl[4-(diphenylsulfoniumyl)phenyl]sulfonium.

Preferred anions AnA⁻ are BF₄⁻, PF₆⁻, AsF₆⁻, SbF₆⁻, ClO₄⁻, Cl⁻, Br⁻, tetraphenylborate, tetrakis(pentafluorophenyl)borate, the benzenesulfonate anion, the p-toluenesulfonate anion and the trifluoromethanesulfonate anion.

Suitable iodonium salts are described for example in DE-A1 197 30 498, particularly on page 3 therein, in lines 40-43, that passage hereby being expressly incorporated into the present disclosure content by reference.

These salts are preferably salts of the formula

R²¹—I⁺—R²² AnB⁻

in which

R²¹ and R²² are optionally substituted aryl groups and AnB⁻ is an anion.

Particular preference is given to diphenyliodonium, anisylphenyliodonium, di(4-tert-butylphenyl)iodonium, di(4-chlorophenyl)iodonium, ditolyliodonium and di(3-nitro-phenyl)iodonium.

Preferred anions AnB⁻ are BF₄⁻, PF₆⁻, AsF₆⁻, SbF₆⁻, ClO₄⁻, Cl⁻, Br⁻, tetraphenylborate, tetrakis(pentafluorophenyl)borate, the benzenesulfonate anion, the p-toluenesulfonate anion and the trifluoromethanesulfonate anion.

Suitable sulfones are described for example in DE-A1 197 30 498, particularly on page 4 therein, in lines 1-12, that passage being hereby expressly incorporated into the present disclosure content by reference.

These sulfones are preferably of the formula

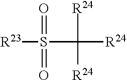

in which

R²³ is an optionally substituted aryl group and the radicals R²⁴ are each a halogen atom.

Halogen for the purposes of this specification comprises fluorine, chlorine, bromine and iodine, preferably chlorine and bromine and more preferably chlorine.

Particular preference is given to trichloromethyl phenyl sulfone, tribromomethyl phenyl sulfone, trichloromethyl 4-chlorophenyl sulfone, tribromomethyl 4-nitrophenyl sulfone, 2-trichloromethylbenzothiazole sulfone, 2,4-dichlorophenyl trichloromethyl sulfone, 2-methyl-4-chlorophenyl trichloromethyl sulfone and 2,4-dichlorophenyl tribromomethyl sulfone.

Suitable peroxides are described for example in DE-A1 197 30 498, particularly on page 4 therein, in lines 13-24, that passage being hereby expressly incorporated into the present disclosure content by reference.

These peroxides are preferably of the formula

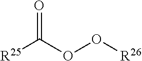

in which

R²⁵ is an optionally substituted aryl group and

R²⁶ is an optionally substituted alkyl group, an optionally substituted aryl group or an optionally substituted benzoyl group, preferably of the formula R²⁵—(CO)—.

Particular preference is given to benzoyl peroxide, 2,4-dichlorobenzoyl peroxide, tert-butyl peroxybenzoate, di(tert-butyl peroxy)isophthalate, di(tert-butyl peroxy)-terephthalate, di(tert-butyl peroxy)phthalate, 2,5-dimethyldi(benzoylperoxy)hexane and 3,3′,4,4′-tetra(tert-butylperoxycarbonyl)benzophenone.

Suitable pyridine N-oxides are described for example in DE-A1 197 30 498, particularly on page 3 therein, in lines 44-62, that passage being hereby expressly incorporated into the present disclosure content by reference.

These N-oxides are preferably of the formula

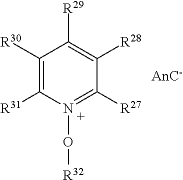

in which

R²⁷, R²⁸, R²⁹, R³⁰ and R³¹ independently of one another are each a hydrogen atom, a halogen atom, a cyano group, an optionally substituted alkyl group, an optionally substituted alkoxy group or an optionally substituted aryl group, R³² is an optionally substituted alkyl group, and AnC⁻ is an anion.

Particular preference is given to N-methoxypyridinium, N-ethoxypyridinium, N-methoxy-2-picolinium, N-methoxy-3-picolinium, N-ethoxy-2-picolinium, N-ethoxy-3-picolinium, N-methoxy-4-bromopyridinium, N-methoxy-3-bromopyridinium, N-methoxy-2-bromopyridinium, N-ethoxy-4-bromopyridinium, N-ethoxy-3-bromopyridinium, N-ethoxy-2-bromopyridinium, N-ethoxy-4-chloropyridinium, N-ethoxy-3-chloropyridinium, N-ethoxy-2-chloropyridinium, N-methoxy-4-methoxypyridinium, N-methoxy-3-methoxypyridinium, N-methoxy-2-methoxypyridinium, N-ethoxy-4-methoxypyridinium, N-ethoxy-3-methoxypyridinium, N-ethoxy-2-methoxypyridinium, N-methoxy-4-phenylpyridinium, N-methoxy-3-phenylpyridinium, N-methoxy-2-phenylpyridinium, N-ethoxy-4-phenylpyridinium, N-ethoxy-3-phenylpyridinium, N-ethoxy-2-phenylpyridinium, N-methoxy-4-cyanopyridinium, N-ethoxy-4-cyanopyridinium, N,N′-dimethoxy-4,4′-bipyridinium, N,N′-diethoxy-4,4′-bipyridinium, N,N′-dimethoxy-2,2′-bipyridinium and N,N′-diethoxy-2,2′-bipyridinium.

Preferred anions AnC⁻ are BF₄⁻, PF₆⁻, AsF₆⁻, SbF₆⁻, ClO₄⁻, Cl⁻, Br⁻, tetraphenylborate, tetrakis(pentafluorophenyl)borate, the benzenesulfonate anion, the p-toluenesulfonate anion and the trifluoromethanesulfonate anion.

Suitable halomethyltriazines are described for example in DE-A1 197 30 498, particularly on page 4 therein, in lines 25-40, that passage being hereby expressly incorporated into the present disclosure content by reference.

These halomethyltriazines are preferably of the formula

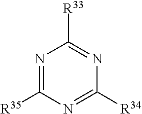

in which

R³³, R³⁴ and R³⁵ independently of one another are each a trihalomethyl group, an optionally substituted alkyl group, an optionally substituted alkenyl group or an optionally substituted aryl group, with the proviso that at least one of the groups is a trihalomethyl group.

Particular preference is given to 2,4,6-tris(trichloromethyl)-s-triazine, 2,4,6-tris(tribromomethyl)-s-triazine, 2,4-bis(dichloromethyl)-6-trichloromethyl-s-triazine, 2-propionyl-4,6-bis(trichloromethyl)-s-triazine, 2-benzoyl-4,6-bis(trichloromethyl)-s-triazine, 2-(4-cyanophenyl-4,6-bis(trichloromethyl)-s-triazine, 2-(4-nitrophenyl)-4,6-bis(trichloromethyl)-s-triazine, 2-(4-chlorophenyl-4,6-bis(trichloromethyl)-s-triazine, 2-(4-cumenyl)-4,6-bis(trichloromethyl)-s-triazine, 2-(4-aminophenyl)-4,6-bis(trichloromethyl)-s-triazine, 2,4-bis(4-methoxyphenyl)-6-trichloromethyl, 2,4-bis(3-chlorophenyl)-6-trichloromethyl-s-triazine, 2-(4-methoxystyryl(trichloromethyl)-s-triazine, 2-(4-chlorostyryl)-4,6-bis(trichloromethyl)-s-triazine, 2-(4-aminophenyl)-4,6-bis(trichloromethyl)-s-triazine, 2,4-bis(4-methoxyphenyl)-6-trichloromethyl-s-triazine, 2,4-bis(3-chlorophenyl)-6-trichloromethyl-s-triazine, 2-(4-methoxystyryl)-4,6-bis(trichloromethyl)-s-triazine, 2-(4-chlorostyryl)-4,6-bis(trichloromethyl)-s-triazine, 2-(4-aminostyryl)-4,6-bis(dichloromethyl)-s-triazine, 2-(4′-methoxy-1′-naphthyl)-4,6-bis(trichloromethyl)-s-triazine and 2-(6′-nitro-1′-naphthyl)-4,6-bis(trichloromethyl)-s-triazine.

The mixture may further comprise at least one solvent (C). These can be, for example, esters, such as butyl acetate or ethyl acetate, aromatic or (cyclo)aliphatic hydrocarbons, such as xylene, toluene or heptane, ketones, such as acetone, isobutyl methyl ketone, methyl ethyl ketone or cyclohexanone, alcohols such as ethanol, isopropanol, mono- or lower oligo-ethylene or -propylene glycols, mono- or dietherified ethylene or propylene glycol ethers, glycol ether acetates, such as methoxypropyl acetate, cyclic ethers such as tetrahydrofuran, carboxamides such as dimethylformamide or N-methylpyrrolidone and/or water, for example.

Preferred mixtures of the invention are composed of
at least one component (A) of the formula An⁻Cya⁺ as indicated above,
at least one component (B), preferably of the formula (IV), having a counterion 1/x cat^{x+}, and
if appropriate at least one solvent (C).

In one preferred embodiment the mixtures of the invention are used without solvent (C).

The weight ratio between component (A) of the formula An⁻ Cya⁺ and component (B) of the formula (IV) with counterion 1/x cat^{x+} in the mixtures of the invention is preferably 1:1 to 1:5, more preferably 1:1 to 1:4, very preferably 1:2 to 1:4.

The mixtures of the invention are highly soluble in coating materials. The solubility can be influenced by the choice of anion and by the choice of the substituents on the cation. Longer alkyl chains as substituents on the cyanine or on the anion of the formula (II) or (III) generally also lead to better solubility.

The sensitizer dyes of the invention generally have absorption maxima in the wavelength range from 400 nm to 650 nm. The absorption maximum of the sensitizer dye can be influenced by the skilled worker in a manner known in principle through the choice of the substituents on the cyanine cation.

The IR radiation used for photocuring can be broadband radiation such as that from light-emitting diodes (LEDs), halogen lamps, Xe lamps, etc. It can also be narrowband radiation or can be laser radiation of a specific wavelength. Particularly suitable lasers are the known lasers which emit in the IR range, examples being semiconductor diode lasers. The radiation may be supplied in a continuous or pulsed form, as in the form of flashes, for example.

The present invention further provides radiation-curable coating materials which comprise the mixtures of the invention.

Coating materials of this kind typically comprise
at least one component (A) of the formula An⁻ Cya⁺ as indicated above,
at least one component (B), preferably of the formula (IV), having a counterion 1/x cat^{x+},
if appropriate at least one solvent (C),
at least one binder (D),
if appropriate at least one reactive diluent (E),
if appropriate at least one UV photoinitiator (F),
if appropriate at least one colorant (G) and
if appropriate further, typical coatings additives (H).

Binders (D) are compounds having free-radically polymerizable ethylenically unsaturated groups. The radiation-curable material comprises preferably 0.001 to 12, more preferably 0.1 to 8 and very preferably 0.5 to 7 mol of radiation-curable ethylenically unsaturated groups per 1000 g of radiation-curable compounds.

Suitable radiation-curable compounds include, for example, (meth)acrylic compounds, vinyl amides, unsaturated polyesters, based for example on maleic acid or fumaric acid, or maleimide/vinyl ether systems.

Preference is given to (meth)acrylate compounds such as polyester (meth)acrylates, polyether (meth)acrylates, urethane (meth)acrylates, epoxy (meth)acrylates, carbonate (meth)acrylates, silicone (meth)acrylates and acrylated polyacrylates.

Preferably at least 40 mol%, more preferably at least 60%, of the radiation-curable ethylenically unsaturated groups are (meth)acrylic groups.

The radiation-curable compounds may be in the form, for example, of a solution, in an organic solvent or water, for example, or in the form of an aqueous dispersion or a powder.

Preference is given to those radiation-curable compounds, and hence also those radiation-curable materials, which are fluid at room temperature. It may, however, also be advantageous to apply the radiation-curable compound or the coating material in the form of a melt or as a powder (powder coating material). The radiation-curable materials comprise preferably less than 20%, in particular less than 10%, by weight of organic solvents and/or water. Preferably they are solvent-free and water-free (i.e., 100% systems). In this case it is possible with preference to dispense with a drying step.

Reactive diluents (E) are, for example, esters of (meth)acrylic acid with alcohols having 1 to 20 carbon atoms, examples being methyl (meth)acrylate, ethyl (meth)acrylate, butyl (meth)acrylate, 2-ethylhexyl (meth)acrylate, 2-hydroxyethyl acrylate, 4-hydroxybutyl acrylate, dihydrodicyclopentadienyl acrylate, vinylaromatic compounds, examples being styrene and divinylbenzene, α,β-unsaturated nitriles, examples being acrylonitrile and methacrylonitrile, α,β-unsaturated aldehydes, examples being acrolein and methacrolein, vinyl esters, examples being vinyl acetate and vinyl propionate, halogenated ethylenically unsaturated compounds, examples being vinyl chloride and vinylidene chloride, conjugated unsaturated compounds, examples being butadiene, isoprene and chloroprene, monounsaturated compounds, examples being ethylene, propylene, 1-butene, 2-butene and isobutene, cyclic monounsaturated compounds, examples being cyclopentene, cyclohexene and cyclododecene, N-vinylformamide, allylacetic acid, vinylacetic acid, monoethylenically unsaturated carboxylic acids having 3 to 8 carbon atoms and also their water-soluble alkali metal, alkaline earth metal or ammonium salts, such as, for example: acrylic acid, methacrylic acid, dimethylacrylic acid, ethacrylic acid, maleic acid, citraconic acid, methylenemalonic acid, crotonic acid, fumaric acid, mesaconic acid and itaconic acid, N-vinylpyrrolidone, N-vinyllactams, an example being N-vinylcaprolactam, N-vinyl-N-alkyl-carboxamides or N-vinylcarboxamides, such as N-vinylacetamide, N-vinyl-N-methylformamide and N-vinyl-N-methylacetamide, or vinyl ethers, examples being methyl vinyl ether, ethyl vinyl ether, n-propyl vinyl ether, isopropyl vinyl ether, n-butyl vinyl ether, sec-butyl vinyl ether, isobutyl vinyl ether, tert-butyl vinyl ether, 4-hydroxybutyl vinyl ether, and also mixtures thereof.

(Meth)acrylic acid stands in this specification for methacrylic acid and acrylic acid, preferably for acrylic acid.

As UV photoinitiators (F) it is possible to use those photoinitiators that are known to the skilled worker, examples being those specified in “Advances in Polymer Science”, Volume 14, Springer Berlin 1974 or in K. K. Dietliker, Chemistry and Technology of UV and EB Formulation for Coatings, Inks and Paints, Volume 3; Photoinitiators for Free Radical and Cationic Polymerization, P. K. T. Oldring (Eds.), SITA Technology Ltd, London. In contrast to the IR photoinitiators, the UV photoinitiators are excited substantially by light in the wavelength range of 2.=200 to 700 nm, more preferably of λ=200 to 500 nm and very preferably of λ=250 to 400 nm.

In accordance with the invention this comprehends those photoinitiators which release free radicals on exposure to light and are able to initiate a free-radical reaction, such as a free-radical polymerization for example.

Suitable examples include phosphine oxides, benzophenones, a-hydroxyalkyl aryl ketones, thioxanthones, anthraquinones, acetophenones, benzoins and benzoin ethers, ketals, imidazoles or phenylglyoxylic acids, and mixtures thereof.

Phosphine oxides are, for example, mono- or bisacylphosphine oxides, as described for example in EP-A 7 508, EP-A 57 474, DE-A 196 18 720, EP-A 495 751 or EP-A 615 980, examples being 2,4,6-trimethylbenzoyldiphenylphosphine oxide, ethyl 2,4,6-trimethylbenzoylphenylphosphinate or bis(2,6-dimethoxybenzoyl)-2,4,4-trimethylpentylphosphine oxide,

benzophenones are, for example, benzophenone, 4-aminobenzophenone, 4,4′-bis(di-methylamino)benzophenone, 4-phenylbenzophenone, 4-chlorobenzophenone, Michler's ketone, o-methoxybenzophenone, 2,4,6-trimethylbenzophenone, 4-methylbenzophenone, 2,4-dimethylbenzophenone, 4-isopropylbenzophenone, 2-chlorobenzophenone, 2,2′-dichlorobenzophenone, 4-methoxybenzophenone, 4-propoxybenzophenone or 4-butoxybenzophenone,

α-hydroxyalkyl aryl ketones are, for example, 1-benzoylcyclohexan-1-ol (1-hydroxy-cyclohexyl phenyl ketone), 2-hydroxy-2,2-dimethylacetophenone (2-hydroxy-2-methyl-1-phenylpropan-1-one), 1-hydroxyacetophenone, 1-[4-(2-hydroxyethoxy)phenyl]-2-hydroxy-2-methyl-1-propan-1-one or a polymer comprising 2-hydroxy-2-methyl-1-(4-isopropen-2-ylphenyl)propan-1-one in copolymerized form

xanthones and thioxanthones are, for example, 10-thioxanthenone, thioxanthen-9-one, xanthen-9-one, 2,4-dimethylthioxanthone, 2,4-diethylthioxanthone, 2,4-diisopropylthioxanthone, 2,4-dichlorothioxanthone or chloroxanthenone,

anthraquinones are, for example, β-methylanthraquinone, tert-butylanthraquinone, anthraquinonecarboxylic esters, benz[de]anthracen-7-one, benz[a]anthracene-7,12-dione, 2-methylanthraquinone, 2-ethylanthraquinone, 2-tert-butylanthraquinone, 1-chloroanthraquinone or 2-amylanthraquinone,

acetophenones are, for example, acetophenone, acetonaphthoquinone, valerophenone, hexanophenone, α-phenylbutyrophenone, p-morpholinopropiophenone, dibenzosuberone, 4-morpholinobenzophenone, p-diacetylbenzene, 4′-methoxyacetophenone, α-tetralone, 9-acetylphenanthrene, 2-acetylphenanthrene, 3-acetylphenanthrene, 3-acetylindole, 9-fluorenone, 1-indanone, 1,3,4-triacetylbenzene, 1-acetonaphthone, 2-acetonaphthone, 2,2-dimethoxy-2-phenylacetophenone, 2,2-diethoxy-2-phenylacetophenone, 1,1-dichloroacetophenone, 1-hydroxyacetophenone, 2,2-diethoxyacetophenone, 2-methyl-1-[4-(methylthio)phenyl]-2-morpholinopropan-1-one, 2,2-dimethoxy-1,2-diphenylethan-2-one or 2-benzyl-2-dimethylamino-1-(4-morpholinophenyl)butan-1-one,

benzoins and benzoin ethers are, for example, 4-morpholinodeoxybenzoin, benzoin, benzoin isobutyl ether, benzoin tetrahydropyranyl ether, benzoin methyl ether, benzoin ethyl ether, benzoin butyl ether, benzoin isopropyl ether or 7H-benzoin methyl ether; or

ketals are, for example, acetophenone dimethyl ketal, 2,2-diethoxyacetophenone, or benzil ketals, such as benzil dimethyl ketal.

Phenylglyoxylic acids are described for example in DE-A 198 26 712, DE-A 199 13 353 or WO 98/33761.

Photoinitiators which can be used additionally are, for example, benzaldehyde, methyl ethyl ketone, 1-naphthaldehyde, triphenylphosphine, tri-o-tolylphosphine or 2,3-butanedione.

Typical mixtures comprise, for example, 2-hydroxy-2-methyl-1-phenylpropan-2-one and 1-hydroxycyclohexyl phenyl ketone, bis(2,6-dimethoxybenzoyl)-2,4,4-trimethyl-pentylphosphine oxide and 2-hydroxy-2-methyl-1-phenylpropan-1-one, benzophenone and 1-hydroxycyclohexyl phenyl ketone, bis(2,6-dimethoxybenzoyl)-2,4,4-trimethyl-pentylphosphine oxide and 1-hydroxycyclohexyl phenyl ketone, 2,4,6-trimethylbenzoyl-diphenylphosphine oxide and 2-hydroxy-2-methyl-1-phenylpropan-1-one, 2,4,6-trimethylbenzophenone and 4-methylbenzophenone, or 2,4,6-trimethylbenzophenone and 4-methylbenzophenone and 2,4,6-trimethylbenzoyldiphenylphosphine oxide.

In one preferred embodiment of the present invention at least one UV photoinitiator is present in the coating materials of the invention.

Colorant (G) is used comprehensively for the purposes of this specification for pigments and dyes, preferably for pigments.

Pigments (G) are, according to CD Römpp Chemie Lexikon—Version 1.0, Stuttgart/New York: Georg Thieme Verlag 1995, with reference to DIN 55943, particulate “organic or inorganic, chromatic or achromatic colorants which are virtually insoluble in the application medium”.

Virtually insoluble here means a solubility at 25° C. of less than 1 g /1000 g of application medium, preferably below 0.5, more preferably below 0.25, very preferably below 0.1 and in particular below 0.05 g /1000 g of application medium.

Examples of pigments in the true sense comprise any desired systems of absorption pigments and/or effect pigments, preferably absorption pigments. The number and selection of the pigment components are not subject to any restrictions whatsoever. They may be adapted to the particular requirements, such as the desired color impression, for example, in an arbitrary way. By way of example it is possible for all of the pigment components of a standardized paint mixer system to be taken as the basis.

By effect pigments are meant all pigments which exhibit a platelet-shaped construction and impart specific decorative color effects to a surface coating. The effect pigments are, for example, all of the effect-imparting pigments which can be employed commonly in vehicle finishing and industrial coating. Examples of effect pigments of this kind are pure metal pigments, such as, for example, aluminum, iron or copper pigments; interference pigments, such as titanium dioxide-coated mica, iron oxide-coated mica, mixed oxide-coated mica (e.g., with titanium dioxide and Fe₂O₃ or titanium dioxide and Cr₂O₃), metal oxide-coated aluminum, or liquid-crystal pigments.

The color-imparting absorption pigments are, for example, customary organic or inorganic absorption pigments which can be used in the paint industry. Examples of organic absorption pigments are azo pigments, phthalocyanine pigments, quinacridone pigments and pyrrolopyrrole pigments. Examples of inorganic absorption pigments are iron oxide pigments, titanium dioxide and carbon black.

Dyes are likewise colorants and differ from the pigments in their solubility in the application medium, i.e., they have a solubility at 25° C. of more than 1 g /1000 g in the application medium.

Examples of dyes are azo, azine, anthraquinone, acridine, cyanine, oxazine, polymethine, thiazine and triarylmethane dyes. These dyes can be employed as basic or cationic dyes, mordant dyes, direct dyes, disperse dyes, developing dyes, vat dyes, metal complex dyes, reactive dyes, acid dyes, sulfur dyes, coupling dyes or substantive dyes.

As further, typical coatings additives (H) it is possible to make use for example of antioxidants, stabilizers, activators (accelerants), fillers, antistats, flame retardants, thickeners, thixotropic agents, surface-active agents, viscosity modifiers, plasticizers or chelating agents.

As accelerants for the thermal aftercure it is possible to use, for example, tin octoate, zinc octoate, dibutyltin laurate or diazabicyclo[2.2.2]octane.

In addition it is possible to add one or more photochemically and/or thermally activable initiators, examples being potassium peroxodisulfate, dibenzoyl peroxide, cyclohexanone peroxide, di-tert-butyl peroxide, azobisisobutyronitrile, cyclohexylsulfonyl acetyl peroxide, diisopropyl percarbonate, tert-butyl peroctoate or benzpinacol, and also, for example, those thermally activable initiators which have a half-life at 80° C. of more than 100 hours, such as di-t-butyl peroxide, cumene hydroperoxide, dicumyl peroxide, t-butyl perbenzoate, silylated pinacols, which are available commercially, for example, under the trade name ADDID 600 from Wacker, or hydroxyl-containing amine N-oxides, such as 2,2,6,6-tetramethylpiperidine-N-oxyl, 4-hydroxy-2,2,6,6-tetra-methyl-piperidine-N-oxyl, etc.

Further examples of suitable initiators are described in “Polymer Handbook”, 2nd ed., Wiley & Sons, New York.

Suitable thickeners, besides free-radically (co)polymerizable (co)polymers, include customary organic and inorganic thickeners such as hydroxymethylcellulose or bentonite.

Examples of chelating agents which can be used include ethylenediamineacetic acid and its salts and also β-diketones.

Coloristically inert fillers are all substances/compounds which on the one hand are coloristically inactive—that is, they exhibit little intrinsic absorption and have a refractive index similar to that of the coating medium—and on the other hand are capable of influencing the orientation (parallel alignment) of the effect pigments in the surface coating, i.e., in the applied paint film, and also properties of the coating or of the coating materials, such as hardness or rheology. Inert substances/compounds which can be used are given by way of example below, but without restricting the concept of coloristically inert, topology-influencing fillers to these examples. Suitable inert fillers meeting the definition may be, for example, transparent or semitransparent fillers or pigments, such as plastic granules, silica gels, blanc fixe, kieselguhr, talc, calcium carbonates, lime, kaolin, barium sulfate, magnesium silicate, aluminum silicate, crystalline silicon dioxide, amorphous silica, aluminum oxide. Additionally as inert fillers it is possible to employ any desired solid inert organic particles, such as urea-formaldehyde condensates, micronized polyolefin wax and micronized amide wax, for example. The inert fillers can in each case also be used in a mixture. It is preferred, however, to use only one filler in each case.

Suitable stabilizers comprise typical UV absorbers such as oxanilides, triazines and benzotriazole and benzophenones. These can be used alone or together with suitable free-radical scavengers, examples being sterically hindered amines such as 2,2,6,6-tetramethylpiperidine, 2,6-di-tert-butylpiperidine or derivatives thereof, e.g., bis(2,2,6,6-tetramethyl-4-piperidyl) sebacate. Stabilizers are customarily used in amounts of 0.1% to 5.0% by weight, based on the solid components comprised in the preparation.

The mixtures of the present invention can be used as IR-activable photoinitiators and exhibit better solubility in coating materials and paint systems than the prior-art formulations of IR photoinitiators in which borate ions of the formula (IV) function as the counterion for the cyanine cation. The consequence of this is that, on the one hand, the photoinitiator can be distributed more uniformly in the paint system, and no undissolved particles remain as defects in the subsequent paint, and that, on the other hand, a higher photoreactivity results. In the prior-art IR photoinitiators the compounds to some extent precipitate as crystals in the coating material, owing to the inadequate solubility.

To achieve this improved solubility the mixtures of the invention are blended with radiation-curable compounds, i.e., for example, binders (D) and/or reactive diluents (E), or preparations comprising them, such as coating materials, paints or paint formulations, for example.

It is a particular advantage of the IR photoinitiators that photoinitiators of this kind are able to initiate a free-radical polymerization even in pigmented paints, since the activating radiation is generally absorbed only little, or not at all, by pigments, whereas the UV radiation required to activate UV photoinitiators is normally absorbed and/or scattered by the pigments and therefore has a low depth of penetration into the coating. Accordingly it is a preferred embodiment of the present invention to use the mixtures of the invention in pigmented coating materials.

A further advantageous embodiment of the present invention involves using the mixtures of the invention in coating materials with high coat thicknesses. Thus in one preferred embodiment the mixtures will be used in coating materials which exhibit a coat thickness of more than 30 μm, preferably more than 45 μm and more preferably more than 60 μm. The coating materials may have a thickness of up to 300 μm, preferably up to 250 μm and more preferably up to 200 μm.

It is of course also possible to apply the coatings more thickly or thinly, at from 10 to 1000 μm for example. In the case of coating materials applied very thickly, however, it may be necessary to irradiate two or more times.

The radiation-curable coating composition may be applied preferably in a simple way as for example by spraying, trowelling, spreading, knife coating, brushing, rolling, roller coating, pouring, dipping, laminating, injection-back molding or coextruding, etc., to the article that is to be coated, and can be dried if appropriate.

Curing is effected by irradiation using electromagnetic radiation which comprises the visible range and the IR range, preferably the visible-light range, and more preferably using electromagnetic radiation in the wavelength range of 400-650 nm.

In one preferred embodiment of the invention the irradiation may also be carried out in the absence of oxygen. For that purpose irradiation is carried out such that at the moment of its irradiation with IR radiation the coating material is exposed to an oxygen partial pressure of less than 18 kPa. The relevant regions are the surface regions of the article to be coated that are provided with the radiation-curable coating materials, at the moment of exposure. Preferably the oxygen partial pressure is not more than 17 kPa, more preferably not more than 15.3 kPa, very preferably not more than 13.5 kPa, in particular not more than 10 kPa, and especially not more than 6.3 kPa.

Complete absence of oxygen is often unnecessary, and so the oxygen partial pressure need not be below, preferably, 0.5 kPa, more preferably 0.9 kPa, very preferably 1.8 kPa, and in particular 2.5 kPa.

One observed advantage of curing under low oxygen partial pressures is, for example, an improved scratch resistance.

A low oxygen partial pressure of this kind can be obtained advantageously by diluting the oxygen-containing atmosphere with at least one inert gas or replacing it by at least one inert gas, in other words gases which are unreactive under the conditions of radiation curing. Suitable inert gases include, preferably, nitrogen, noble gases, carbon dioxide or combustion gases. In the atmosphere under which the radiation cure is carried out, the fraction of said at least one inert gas ought to be more than 80% by volume, preferably at least 85%, more preferably at least 90%, very preferably at least 95%, and in particular at least 98% by volume. Irradiation may additionally take place with the coating material covered by transparent media. Transparent media are, for example, polymeric films, glass or liquids, e.g., water. Irradiation takes place with particular preference as described in WO 01/14483, hereby incorporated in its entirety by reference. Very particular preference is given to irradiation in the manner described in DE-A1 199 57 900, hereby incorporated in its entirety by reference.

The coating materials and paint formulations of the invention are especially suitable for coating substrates such as wood, preferably pine, fir, beech, oak or maple wood, paper, cardboard, paperboard, textile, leather, leather substitutes, nonwovens, plastics surfaces, preferably SAN, PMMA, ABS, PP, PS, PC or PA (abbreviations according to DIN 7728), glass, ceramic, mineral building materials, such as cement moldings and fiber-cement slabs, or uncoated or coated metals, preferably plastics or metals, which may also be in the form of sheets (foils or films), for example. The coated or uncoated metal may also, for example, be shaped into rolls, referred to as coils, for the purpose of storage or of transport. The coating of the metals may comprise customary primer coatings or a cathodic deposition coating.

With particular preference the coating materials of the invention are suitable for outdoor applications or in applications involving exposure to daylight, preferably of buildings or parts of buildings, for interior coatings, and coatings on aircraft and vehicles. In particular the coating materials of the invention are used as or in automotive clearcoat and topcoat material(s) and also in paints, especially masonry paints, industrial coatings, coil coatings, molding compounds, casting compounds or dental compounds. A further possibility is to use the mixtures of the invention to cure building materials, tiles, clinker, artificial stone, screeding, plasters and coating materials for the purpose of their coating. The coating materials of the invention are useful with advantage for decorative coating, particularly for coating furniture, wood-block flooring, laminate and floor coverings.

It is possible, furthermore, to use the coating materials of the invention in printing processes or for producing printing plates, as for example in stereolithography, photolithography, in screen printing, offset, planographic printing, gravure printing or relief printing processes, and also in the ink-jet process.

The examples which follow are intended to illustrate the properties of the invention, but without restricting it.

By “parts” or “%” are meant in this specification, unless indicated otherwise, “parts by weight” or “% by weight”.

### EXAMPLE 1

### 2-((E)-2-{4-[Bis(2-cyanoethyl)amino]phenyl}vinyl)-1,3,3-trimethyl-3H-indolium-4-dodecylbenzenesulfonate (Table No. 20):

The synthesis was carried out in an automatic Labvision® Laboratory unit which consisted essentially of two jacketed vessels, 250 ml and 2500 ml, connected by a glass bridge. The two vessels were equipped with glass impeller stirrers, flow disruptors and thermostats. The rotary speed was 350 rpm in the 250 ml vessel and 250 rpm in the 2500 ml vessel.

First 100 g of dimethylformamide (DMF) were charged to the 250 ml vessel, 100 g of N,N-bis(cyanoethyl)aniline were added, and a rinse with 25.5 g of DMF was carried out. Then the thermostat was set to 20° C. and 80 g of phosphorus oxychloride (POCl₃) were metered in over the course of 40 minutes. After the end of metering the batch was heated to 90° C. and stirred for 1 hour.

During the heating operation in the small vessel, the large vessel was charged with 300 g of water and the thermostat was set at 40° C. Following complete conversion in the small vessel, the hydrolysis was carried out—the bottom drain in the small vessel was slowly opened and the reaction solution, which had a temperature of 90° C., ran through the glass bridge into the water-filled large vessel. After about 5 minutes a fine, pale yellow solid was precipitated. The small vessel was rinsed with 10-15 ml of DMF and stirring was carried out for 5 minutes. Then 400 g of diethyl ketone (DEK) were metered in in the large vessel, and, after the end of the addition, the contents were heated to 70° C. At 70° C. a pH of 6.0 was set with 50% strength aqueous sodium hydroxide solution over the course of 15 minutes. This was followed by an hour of stirring at 70° C., after which phase separation took place. The water phase was discarded. The organic phase, which had a temperature of 70° C., was admixed rapidly at 200 rpm with 88 g of 1,3,3-trimethyl-2-methyleneindoline (tribase) and then, over the course of 6 minutes, with 52 g of concentrated sulfuric acid. The batch was then heated to 100° C. and water of reaction was removed by distillation. Stirring was continued at 100° C. for about an hour, during which water of reaction was still distilled off. Thereafter the reaction mixture was cooled to 90° C. and a solution of 184 g of sodium dodecylbenzenesulfonate in 800 g of water (temperature of the solution: approximately 60° C.) was added. Stirring was continued at 70° C. for 1.75 hours. Following further phase separation, the organic phase was concentrated under reduced pressure. This gave 330 g (92% yield over 3 stages) of a red solid.

### EXAMPLE 2

### 2-((E)-2-{4-[(2-Cyanoethyl)(2-hydroxyethyl)amino]-2-methylphenyl}vinyl)-1-ethyl-3,3-dimethyl-3H-indolium 4-dodecylbenzenesulfonate (Table No. 10)

Vielsmeier-Haack Formylation:

A 250 ml four-neck flask with nitrogen flush, reflux condenser and KPG stirrer was charged with 8 g of dimethylformamide and 8.6 g of 2-[(2-cyanoethyl)m-tolylamino]-ethylacetic ester. At 5° to 10° C., 6 g of phosphorus oxychloride were added dropwise over 10 minutes. Thereafter the mixture was heated to room temperature, stirred at 70° C. for 5.5 hours and left to cool overnight with stirring. After that, 200 ml of methyl tert-butyl ether were added and the mixture was cooled with an ice-water bath, and over 5 minutes at a temperature of not more than 10° C. a solution of 17 g of sodium acetate and 70 ml of water was added dropwise. This was followed by phase separation at room temperature. The organic phase was washed with water and then with saturated sodium hydrogencarbonate solution, dried over sodium sulfate and concentrated on a rotary evaporator.

This gave 8.7 g (91% yield) of 2-[(2-cyanoethyl)(4-formyl-3-methylphenyl)amino]ethyl-acetic ester.

Acetate Deprotection:

A 500 ml Erlenmeyer flask with stirring bar was charged with 120 ml of methanol and 8.7 g of 2-[(2-cyanoethyl)(4-formyl-3-methylphenyl)amino]ethylacetic ester. 40 ml of water and 3.4 g of sodium carbonate were added and the mixture was stirred overnight at room temperature. The next morning it was filtered and the mother liquor was adjusted to a pH of 7. Using a rotary evaporator, methanol was removed from this solution, extraction was carried out three times with dichloromethane, and the combined organic phases were dried over sodium sulfate. Removal of the organic solvent under reduced pressure gave 6.6 g (90% yield) of 3-[(4-formyl-3-methylphenyl)-(2-hydroxyethyl)amino]propionitrile.

Condensation:

In a 250 ml four-neck flask with nitrogen flush, reflux condenser and KPG stirrer, 3.2 g of 3-[(4-formyl-3-methylphenyl)(2-hydroxyethyl)amino]propionitrile and 4.2 g of 1-ethyl-2,3,3-trimethylindolium iodide were suspended in 20 ml of toluene and 6 ml of n-butanol and the suspension was heated to 105° C. and stirred for 2 hours. Overnight it was cooled to room temperature and the resulting suspension was filtered. The filter cake was washed with toluene and methyl tert-butyl ether and dried under reduced pressure at 50° C.

This gave 6.7 g (95% yield) of 2-((E)-2-{4-[(2-cyanoethyl)(2-hydroxyethyl)aminol-2-methylphenyl}vinyl)-1-ethyl-3,3-dimethyl-3H-indolium iodide as a red solid.

Salt Conversion:

In a 1000 ml Erlenmeyer flask with stirring bar, 3 g of 2-((E)-2-{4-[(2-cyanoethyl)-(2-hydroxyethyl)amino]-2-methylphenyl}vinyl)-1-ethyl-3,3-dimethyl-3H-indolium iodide were dissolved in 300 ml of dichloromethane; 2.1 g of sodium dodecylbenzene-sulfonate in 100 ml of water were added, and the mixture was stirred at room temperature for 3 hours. This was followed by phase separation, and the organic phase was dried over sodium sulfate, filtered, concentrated under reduced pressure and dried.

This gave 4 g (97% yield) of 2-((E)-2-{4-[(2-cyanoethyl)(2-hydroxyethyl)amino]-2-methylphenyl}vinyl)-1-ethyl-3,3-dimethyl-3H-indolium 4-dodecylbenzenesulfonate as a red solid.

In the same way as for Example 2, the following dyes were prepared from the corresponding reactants: No. 5, 6 and 7 of the table.

In the same way as for Example 2 but without acetate deprotection, the following dyes were prepared from the corresponding reactants: No. 2, 3, 8, 9, 11, 12 and 19 of the table.

In the same way as for the condensation and salt conversion from Example 2, the following dyes were prepared from the corresponding reactants: No. 1, 4, 13, 14 and 15 of the table.

Starting from (2-((E)-2-{4-[(2-chloroethyl)ethylamino]-2-methylphenyl}vinyl)-1,3,3-trimethyl-3H-indolium chloride (Astrazon red 6 B, source ABCR) and, respectively, (2-((E)-2-{4-[(2-chloroethyl)methylamino]phenyl}vinyl)-1,3,3-trimethyl-3H-indolium chloride (Astrazon pink FG, source ABCR), salt conversion in the same way as for Example 2 gives No. 17 and 18 of the table.

The photoinitiators were assessed with the aid of the following accelerated test:

The test medium used for free-radically polymerizable paint base materials was composed of three different commercial, acrylate-containing paint base materials from BASF SE, Ludwigshafen, of the acrylate type: Laromer® 8863 (polyether acrylate based on ethoxylated trimethylolpropane), Laromer® PO 84 F (amine-modified polyether acrylate based on alkoxylated trimethylolpropane) and Laromer® 8987 (urethane acrylate based on hexamethylene diisocyanate, as a solution in hexanediol diacrylate). The photoinitiators under test were each weighed out into these test media and dissolved as homogenously as possible. The standard concentrations used were 0.5% of sensitizer dye and 1.5% of coinitiator (B) (in general a boranate salt, in particular an n-butyl triphenylboranate salt). The incorporation and the handling of the products took place largely without direct light irradiation.

The paint base material doped with photoinitiator was then applied as a thin coat to a glass support. 1-3 drops of the doped paint base material were applied to a glass slide, two spacer films (approximately 50 μm) were placed alongside it, and, finally, the liquid was covered with a further glass slide; the two slides were pressed together by means of two clips. These “sandwich samples” were then exposed. Measurements on cured coating films gave film thicknesses of approximately 56 μm.

Particularly suitable for the curing of the above-described samples of the doped paint base materials were halogen lamps on account of the particular spectral sensitivity of the photoinitiator systems under investigation here. In the case of the standard investigations, a slide projector (halogen lamp) was used, and the samples were exposed at defined distances with defined exposure times.

The success in photocuring was first evaluated qualitatively for all of the samples: this was done by opening the sandwich samples, by removing the lid, and evaluating the coating film for its hardness, if appropriate by rubbing with a metal spatula, as follows: “still liquid”, “cured—but soft”, “hard”.

Also assessed was the degree of residual color after curing. Here it was found that when using certain dyes, described in Examples 1 and 2, more residual color remained in the coating material when the counterion of the dye was an iodide than when the counterion was an arylsulfonate substituted by a long-chain alkyl radical. For example, the coating materials with dyes 16 and 19 from the table above were almost colorless after curing. The corresponding iodides, in contrast, still exhibited a slight light blue or slight red color, respectively.

### EXAMPLE 3

Cyanine cations exhibit increased photoactivity with arylsulfonates of the formula (II) substituted by long-chain alkyl radicals as counterions, as compared with those containing, as their counterions, alkylsulfonate anions, as known from EP 1091247 A2.

The measure for this was the time in seconds required, under irradiation with a 50 W halogen lamp, to obtain tack-free coatings of clearcoats with a film thickness of 50 μm. For this purpose, mixtures of cyanine cations with dodecylsulfonate (comparison according to EP 1091247 A2) or with dodecylbenzenesulfonate (inventive) with commercial, radiation-curable coating components (Laromer® PO84F, Laromer® 8863, Laromer® 8987 from BASF AG, Ludwigshafen) were prepared and cured.

The results are as follows:

| No. | Structure | Laromer PO84F | Laromer 8863 | Laromer 8987 |
|---|---|---|---|---|
| Ex. 3 | 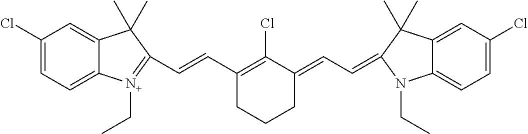 | 1 | 1 | 1 |
| | 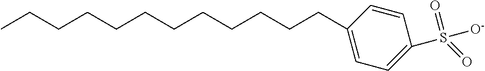 | | | |
| Comp. Ex. 3 | 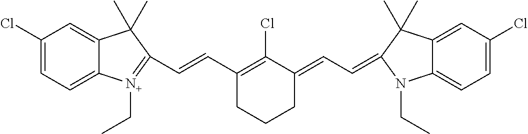 | 60 | 60 | 30 |
| | 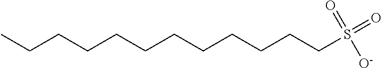 | | | |
| Ex. 4 | 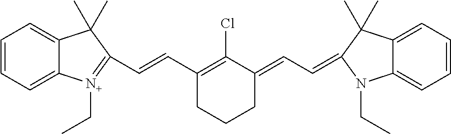 | 32 | 32 | 32 |
| | 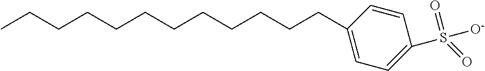 | | | |
| Comp. Ex. 4 | 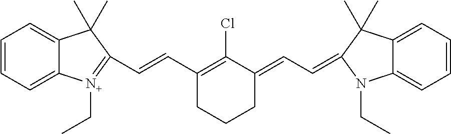 | >60 | >60 | >60 |
| | 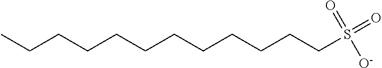 | | | |

It is seen that the cyanine cations which have arylsulfonates substituted by long-chain alkyl radicals as their counterions have a shorter time until tack-free curing of the films is achieved, and hence exhibit a greater photoactive ability, than dodecylsulfonate.

1. A sensitizer system, comprising:
a styrylic cation, D⁺, of formula (I) 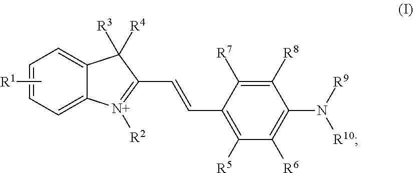
and
as counterion, an anion An^{- selected from the group consisting of} 4-hexylbenzenesulfonate, 4-octylbenzenesulfonate, 4-decylbenzenesulfonate, and 4-dodecylbenzenesulfonate,
wherein
R¹, R⁵, R⁶, R⁷, and R⁸ are each independently hydrogen, C₁-C₁₈ alkyl, or C₁-C₁₈ alkyloxy,
or R¹ is a halogen,
R², R³, and R⁴ are each independently C₁-C₁₈ alkyl,
R⁹ and R¹⁰ are each independently unsubstituted or aryl-, alkyl-, aryloxy-, alkyloxy-, heteroatom- and/or heterocycle-substituted C₁-C₁₈ alkyl, C₆-C₁₂ aryl, or C₅-C₁₂ cycloalkyl,
wherein the sensitizer system is suitable for radiation-curable coating material.

2. The sensitizer system of claim 1, wherein R⁹ and R¹⁰ are each independently unsubstituted or aryl-, alkyl-, aryloxy-, alkyloxy-, heteroatom- and/or heterocycle-substituted C₁-C₁₈ alkyl.

3. The sensitizer system of claim 1, wherein R⁹ and R¹⁰ are each independently selected from the group consisting of methyl, ethyl, n-propyl, 2-hydroxyethyl, 2-hydroxypropyl, 2-chloroethyl, 2-cyanoethyl, 2-acetoxyethyl, cyclohexyl, and cyclopentyl.

4. The sensitizer system of claim 1, wherein the styrylic cation is a compound selected from the group consisting of:
compound 1, wherein R¹, R⁵, R⁶, R⁷, and R⁸ are H, R² is ethyl, R³ and R⁴ are methyl, and R⁹+R¹⁰ is 1,4-butylene;
compound 2, wherein R¹, R⁵, and R⁷ are H, R² is ethyl, R³ and R⁴ are methyl, R⁶+R¹⁰, is 1,3-propylene, and R⁸+R⁹ is 1,3-propylene;
compound 3, wherein R¹, R⁵, R⁶, R⁷, and R⁸ are H, R² is ethyl, R³ and R⁴ are methyl, and R⁹ and R¹⁰ are 2-chloroethyl;
compound 4, wherein R¹, R⁵, R⁶, R⁷, and R⁸ are H, R² is ethyl, R³, R⁴, and R¹⁰ are methyl, and R⁹ is 2-hydroxyethyl;
compound 5, wherein R¹, R⁵, R⁶, R⁷, and R⁸ are H, R² is ethyl, R³ and R⁴ are methyl, and R⁹ and R¹⁰ are 2-hydroxyethyl;
compound 6, wherein R¹, R⁵, R⁶, R⁷, and R⁸ are H, R² is ethyl, R³ and R⁴ are methyl, R⁹ is 2-hydroxyethyl, and R¹⁰ is cyclohexyl;
compound 7, wherein R¹, R⁵, R⁶, R⁷, and R⁸ are H, R² is ethyl, R³ and R⁴ are methyl, R⁹ is 2-hydroxyethyl, and R¹⁰ is 2-cyanoethyl;
compound 8, wherein R¹, R⁵, R⁶, R⁷, and R⁸ are H, R² is ethyl, R³ and R⁴ are methyl, and R⁹ and R¹⁰ are 2-acetoxyethyl;
compound 9, wherein R¹, R⁵, R⁶, R⁷, and R⁸ are H, R² is ethyl, R³, R⁴, and R¹⁰ are methyl, and R⁹ is 2-cyanoethyl;
compound 10, wherein R¹, R⁶, R⁷, and R⁸ are H, R² is ethyl, R³, R⁴, and R⁵ are methyl, R⁹ is 2-hydroxyethyl, and R¹⁰ is 2-cyanoethyl;
compound 11, wherein R¹, R⁶, R⁷, and R⁸ are H, R², R⁹, and R¹⁰ are ethyl, and R³, R⁴, and R⁵ are methyl;
compound 12, wherein R¹, R⁶, R⁷, and R⁸ are H, R² is ethyl, and R³, R⁴, R⁵, R⁹, and R¹⁰ are methyl;
compound 13, wherein R¹, R⁶, R⁷, and R⁸ are H, R² is ethyl, R³, R⁴, and R⁵ are methyl, and R⁹+R¹⁰ is pentylene;
compound 14, wherein R¹, R⁶, R⁷, and R⁸ are H, R² is ethyl, R³, R⁴, and R⁵ are methyl, and R⁹+R¹⁰ is 3-oxa-1,5-pentylene;
compound 15, wherein R¹, R⁵, R⁶, R⁷, and R⁸ are H, R² is ethyl, R³ and R⁴ are methyl, and R⁹+R¹⁰ is 3-oxa-1,5-pentylene;
compound 16, wherein R¹, R⁵, R⁶, R⁷, and R⁸ are H, R² is ethyl, and R³, R⁴, R⁹, and R¹⁰ are methyl;
compound 17, wherein R¹, R⁵, R⁶, R⁷, and R⁸ are H, R², R³, R⁴, and R⁹ are methyl, and R is 2-chloroethyl;
compound 18, wherein R¹, R⁶, R⁷, and R⁸ are H, R⁹ is ethyl, R², R³, R⁴, and R⁵ are methyl, and R¹⁰ is 2-chloroethyl;
compound 19, wherein R¹, R⁵, R⁶, R⁷, and R⁸ are H, R² is ethyl, R³ and R⁴ are methyl, and R⁹ and R¹⁰ are 2-cyanoethyl; and
compound 20, wherein R¹, R⁵, R⁶, R⁷, and R⁸ are H, R², R³, and R⁴ are methyl, and R⁹ and R¹⁰ are 2-cyanoethyl.

5. A mixture, comprising:
at least one component (A), comprising the sensitizer system of claim 1; and
at least one component (B) having a counterion 1/x cat^{x+},
wherein component (B) is selected from the group consisting of tetra-n-octylammonium, tetramethylammonium, tetraethylammonium, tetra-n-butylammonium, trimethylbenzylammonium, trimethylcetylammonium, triethylbenzylammonium, tri-n-butylbenzylammonium, trimethylethylammonium, tri-n-butylethylammonium, triethylmethylammonium, tri-n-butylmethylammonium, diisopropyldiethylammonium, diisopropylethylmethylammonium, diisopropylethylbenzylammonium, N,N-dimethylpiperidinium, N,N-dimethylmorpholinium, N,N-dimethylpiperazinium and N-methyldiazabicyclo[2.2.2]octane.

6. The mixture of claim 5, wherein the cation, cat^{x+}, is selected from the group consisting of 1-methylimidazolium, 1-butylimidazolium, 1,3-dimethylimidazolium, 1,2,3-trimethylimidazolium, 1-n-butyl-3-methylimidazolium, 1-ethyl-3-methylimidazolium, 1,3,4,5-tetramethylimidazolium, 1,3,4-trimethylimidazolium, 2,3-dimethylimidazolium, 1-butyl-2,3-dimethylimidazolium, 3,4-dimethylimidazolium, 2-ethyl-3,4-dimethylimidazolium, 3-methyl-2-ethylimidazolium, 3-butyl-1-methylimidazolium, 3-ethyl-1-methylimidazolium, 3-butyl-1-ethylimidazolium, 3-butyl-1,2-dimethylimidazolium, 1,3-di-n-butylimidazolium, 3-butyl-1,4,5-trimethylimidazolium, 3-butyl-1,4-dimethylimidazolium, 3-butyl-2-methylimidazolium, 1,3-dibutyl-2-methylimidazolium, 3-butyl-4-methylimidazolium, 3-butyl-2-ethyl-4-methylimidazolium, 3-butyl-2-ethylimidazolium, 1-methyl-3-octylimidazolium and 1-decyl-3-methylimidazolium.

7. The mixture of claim 5, wherein a weight ratio between component (A) and component (B) is 1:1 to 1:5.

8. A radiation-curable coating material, comprising:
(A) at least one component formula An⁻ Cya⁺, comprising the sensitizer system of claim 1;
(B) at least one component having a counterion 1/ₓ cat^{x+} selected from the group consisting of tetra-n-octylammonium, tetramethylammonium, tetraethylammonium, tetra-n-butylammonium, trimethylbenzylammonium, trimethylcetylammonium, triethylbenzylammonium, tri-n-butylbenzylammonium, trimethylethylammonium, tri-n-butylethylammonium, triethylmethylammonium, tri-n-butylmethylammonium, diisopropyldiethylammonium, diisopropylethylmethylammonium, diisopropylethylbenzylammonium, N,N-dimethylpiperidinium, N,N-dimethylmorpholinium, N,N-dimethylpiperazinium, and N-methyldiazabicyclo[2.2.2loctane;
(C) optionally, at least one solvent;
(D) at least one binder;
(E) optionally, at least one reactive diluent;
(F) optionally, at least one UV photoinitiator;
(G) optionally, at least one colorant; and
(H) optionally, at least one further (H) coating additive.

9. A method of coating a substrate, comprising:
applying the radiation-curable coating material of claim 8 in a thickness of 10 to 1000 μm to an article to be coated;
optionally, drying; and then
irradiating with electromagnetic radiation in a wavelength range of 700-900 nm.

10. The method of claim 9, wherein the article to be coated is wood, paper, cardboard, paperboard, textile, leather, a leather substitute, a nonwoven, a plastic surface, glass, ceramic, a mineral building material, a coated metal, or an uncoated metal.

11. An automotive clearcoat or topcoat materials material, a paint, an industrial coating, a coil coating, a molding compound, a casting compound, or a dental compound, comprising the radiation-curable coating material of claim 8, in cured or uncured form.

12. The A sensitizer system of claim 1, wherein, in thea styrylic cation, of formula (I), R¹ is bromine.

13. The sensitizer system of claim 1, wherein the styrylic cation is a compound selected from the group consisting of:
compound 1, wherein R¹, R⁵, R⁶, R⁷, and R⁸ are H, R² is ethyl, R³ and R⁴ are methyl, and R⁹+R¹⁰ is 1,4-butylene;
compound 2, wherein R¹, R⁵, and R⁷ are H, R² is ethyl, R³ and R⁴ are methyl, R⁶+R¹⁰, is 1,3-propylene, and R⁸+R⁹ is 1,3-propylene; and
compound 3, wherein R¹, R⁵, R⁶, R⁷, and R⁸ are H, R² is ethyl, R³ and R⁴ are methyl, and R⁹ and R¹⁰ are 2-chloroethyl.

14. The sensitizer system of claim 1, wherein the styrylic cation is a compound selected from the group consisting of:
compound 4, wherein R¹, R⁵, R⁶, R⁷, and R⁸ are H, R² is ethyl, R³, R⁴, and R¹⁰ are methyl, and R⁹ is 2-hydroxyethyl;
compound 5, wherein R¹, R⁵, R⁶, R⁷, and R⁸ are H, R² is ethyl, R³ and R⁴ are methyl, and R⁹ and R¹⁰ are 2-hydroxyethyl; and
compound 6, wherein R¹, R⁵, R⁶, R⁷, and R⁸ are H, R² is ethyl, R³ and R⁴ are methyl, R⁹ is 2-hydroxyethyl, and R¹⁰ is cyclohexyl.

15. The sensitizer system of claim 1, wherein the styrylic cation is a compound selected from the group consisting of:
compound 7, wherein R¹, R⁵, R⁶, R⁷, and R⁸ are H, R² is ethyl, R³ and R⁴ are methyl, R⁹ is 2-hydroxyethyl, and R¹⁰ is 2-cyanoethyl;
compound 8, wherein R¹, R⁵, R⁶, R⁷, and R⁸ are H, R² is ethyl, R³ and R⁴ are methyl, and R⁹ and R¹⁰ are 2-acetoxyethyl; and
compound 9, wherein R¹, R⁵, R⁶, R⁷, and R⁸ are H, R² is ethyl, R³, R⁴, and R¹⁰ are methyl, and R⁹ is 2-cyanoethyl.

16. The sensitizer system of claim 1, wherein the styrylic cation is a compound selected from the group consisting of:
compound 10, wherein R¹, R⁶, R⁷, and R⁸ are H, R² is ethyl, R³, R⁴, and R⁵ are methyl, R⁹ is 2-hydroxyethyl, and R¹⁰ is 2-cyanoethyl;
compound 11, wherein R¹, R⁶, R⁷, and R⁸ are H, R², R⁹, and R¹⁰ are ethyl, and R³, R⁴, and R⁵ are methyl; and
compound 12, wherein R¹, R⁶, R⁷, and R⁸ are H, R² is ethyl, and R³, R⁴, R⁵, R⁹, and R¹⁰ are methyl.

17. The sensitizer system of claim 1, wherein the styrylic cation is a compound selected from the group consisting of:
compound 13, wherein R¹, R⁶, R⁷, and R⁸ are H, R² is ethyl, R³, R⁴, and R⁵ are methyl, and R⁹+R¹⁰ is 1,5-pentylene; and
compound 14, wherein R¹, R⁶, R⁷, and R⁸ are H, R² is ethyl, R³, R⁴, and R⁵ are methyl, and R⁹+R¹⁰ is 3-oxa-1,5-pentylene.

18. The sensitizer system of claim 1, wherein the styrylic cation is a compound selected from the group consisting of:
compound 15, wherein R¹, R⁵, R⁶, R⁷, and R⁸ are H, R² is ethyl, R³ and R⁴ are methyl, and R⁹+R¹⁰ is 3-oxa-1,5-pentylene; and
compound 16, wherein R¹, R⁵, R⁶, R⁷, and R⁸ are H, R² is ethyl, and R³, R⁴, R⁹, and R¹⁰ are methyl.

19. The sensitizer system of claim 1, wherein the styrylic cation is a compound selected from the group consisting of:
compound 17, wherein R¹, R⁵, R⁶, R⁷, and R⁸ are H, R², R³, R⁴, and R⁹ are methyl, and R¹⁰ is 2-chloroethyl; and
compound 18, wherein R¹, R⁶, R⁷, and R⁸ are H, R⁹ is ethyl, R², R³, R⁴, and R⁵ are methyl, and R¹⁰ is 2-chloroethyl.

20. The sensitizer system of claim 1, wherein the styrylic cation is a compound selected from the group consisting of:
compound 19, wherein R¹, R⁵, R⁶, R⁷, and R⁸ are H, R² is ethyl, R³ and R⁴ are methyl, and R⁹ and R¹⁰ are 2-cyanoethyl; and
compound 20, wherein R¹, R⁵, R⁶, R⁷, and R⁸ are H, R², R³, and R⁴ are methyl, and R⁹ and R¹⁰ are 2-cyanoethyl
